(19) United States
(12) Patent Application Publication
(10) Pub. No.: US 2022/0373534 A1
(43) Pub. Date: Nov. 24, 2022

(54) HUMAN NASOPHARYNGEAL CARCINOMA CELLS DERIVED FROM PATIENT DERIVED XENOGRAFT AND USES THEREOF

(71) Applicant: Chang Gung Memorial Hospital, Linkou, Taoyuan City (TW)

(72) Inventor: Cheng-Lung HSU, Taoyuan City (TW)

(21) Appl. No.: 17/749,273

(22) Filed: May 20, 2022

Related U.S. Application Data
(60) Provisional application No. 63/191,099, filed on May 20, 2021.

(51) Int. Cl. G01N 33/50; C12N 5/09

(52) U.S. Cl. CPC G01N 33/5011; C12N 5/0693

(57) ABSTRACT

The present invention provides a human nasopharyngeal carcinoma cell line derived from a patient derived xenograft. The novel human nasopharyngeal carcinoma cells comprise human herpesvirus 4 and specific short tandem repeat loci. Also provided are cellular composition comprising the novel nasopharyngeal carcinoma cell line described herein and the use of the novel nasopharyngeal carcinoma cell line for detecting a potential therapeutic agent.

### CROSS-REFERENCE TO RELATED APPLICATION

This application claims the benefit of U.S. Application No. 63/191,099, filed on 20 May 2022, the entire disclosure of which is incorporated herein by reference.

### BACKGROUND OF THE INVENTION

The subject matter discussed in the background section should not be assumed to be prior art merely as a result of its mention in the background section. Similarly, a problem and the understanding of the causes of a problem mentioned in the background section should not be assumed to have been previously recognized in the prior art. The subject matter in the background section may merely represents different approaches, which in and of themselves may also be inventions.

Nasopharyngeal carcinoma (NPC) is a prevalent head and neck tumor in Southeast Asia, including Taiwan. Individual genetic susceptibility, Epstein—Barr virus (EBV) infection, and dietary or chemical carcinogens are the main etiological factors contributing to NPC pathogenesis. The majority of patients with NPC are aged between 40 and 60 years, and NPC has been rarely diagnosed in patients aged below 30 years and above 70 years. Adolescents and young adults with cancer are likely to have poor outcomes because of the molecularly distinct tumor signature, predisposition to genetic mutations, and severe clinical manifestations. Older patients with NPC often have poor prognosis due to a more advanced cancer stage, complications and side effect of the therapy.

EBV (+) patient-derived xenografts (PDXs), given their close resemblance with patient tumors, serve as important models in preclinical evaluation for novel therapeutic drugs. Although EBV is a crucial viral factor in NPC pathogenesis and progression, most of the available isolated NPC cells have lost the EBV genome during long-term passaging, hindering the progress in this field. Clearly, the screening and evaluation of novel and potentially effective therapeutic agents against NPC is significantly limited by the lack of a preclinical EBV (+) NPC PDX and PDX-derived EBV (+) NPC cells, as EBV (+) NPC PDX and PDX-derived EBV (+) NPC cells are difficult to establish in vivo. The nt invention addresses this need and oth needs.

### BRIEF SUMMARY OF THE INVENTION

In one embodiment, the present invention discloses one or more human nasopharyngeal carcinoma cells derived from a PDX, said cells comprise human herpesvirus 4 (HHV-4) or EBV and at least one of the following short tandem repeat locus: D5S818, D13S317, D7S820, D16S539, vWA, THO1, Amelogenin, PDX, CSF1PO, D3S1358, D1S1656, D2S441, D10S1248, Penta E, D18S51, D2S1338, Penta D, D21S11, DYS19, D8S1179, D12S391, D19S433, FGA or D22S1045.

In another embodiment, the present invention discloses a cellular composition comprising one or more PDX derived human nasopharyngeal carcinoma cells described herein.

Also provided are in vitro methods for identifying a test therapeutic agent to inhibit NPC cells, comprising the steps of : a) contacting the test therapeutic agent with a plurality of NPC cells described herein; and b) determining the quantify of NPC cells or the half maximal inhibitory concentration level (IC50) of the test therapeutic agent, wherein after contacting the test therapeutic agent with the NPC cells described herein, the quantify of NPC cells remains the same or reduced compared to the quantify of NPC prior to the contact, or the therapeutic effective IC50 is reached, is an indication that the test therapeutic agent is efficacious for treating NPC.

The terms “invention,” “the invention,” “this invention” and “the present invention” used in this patent are intended to refer broadly to all of the subject matter of this patent and the patent claims below. Statements containing these terms should be understood not to limit the subject matter described herein or to limit the meaning or scope of the patent claims below. Embodiments of the invention covered by this patent are defined by the claims below, not this summary. This summary is a high-level overview of various aspects of the invention and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification, any or all drawings and each claim.

The invention will become more apparent when read with the accompanying figures and detailed description which follow.

### BRIEF DESCRIPTION OF THE DRAWINGS

Illustrative embodiments of the present invention are described in detail below with reference to the following Figures:

FIG. 1A is a phase microscopic image (400X) and FIG. 1B is an EBER staining image of the PDX derived NPC cells of the present invention.

FIG. 2 shows the Western blot analysis of EGRF expression of the PDX derived NPC cells of the present invention (lane 2-7) and other NPC cell lines (lane 8-11).

FIG. 3A and FIG. 3B are line graphs showing the IC50 of afatinib and palbociclib respectively in PDX derived NPC cells of the present invention.

FIG. 4 is a line graph illustrating the effect of afatinib (AFA), palbociclib (PAL), erbitux (ERB) and combination thereof on the PDX derived NPC cells of the present invention.

### DETAILED DESCRIPTION OF THE INVENTION

As used herein, the articles “a” and “an” refer to one or more than one (i.e., at least one) of the grammatical object of the article.

All numbers herein may be understood as modified by “about”, which is defined as ±10%.

The present invention discloses a plurality of human nasopharyngeal carcinoma cells derived from a patient derived xenograft (PDX), said cells comprise (a) human herpesvirus 4 (HHV-4) or EBV and (b) at least one of the following short tandem repeat locus: D5S818, D13S317, D7S820, D16S539, vWA, THO1, Amelogenin, PDX, CSF1PO, D3S1358, D1S1656, D2S441, D10S1248, Penta E, D18S51, D2S1338, Penta D, D21S11, DYS19, D8S1179, D12S391, D19S433, FGA or D22S1045.

The terms “human herpesvirus 4 or HHV-4” or “Epstein—Barr virus or EBV” are interchangeable. The EBV within the PDX derived human nasopharyngeal carcinoma cells of the present invention can be DNA, RNA, the virus or any combination thereof. The presence of EBV in the PDX derived human nasopharyngeal carcinoma cells can be detected by any known methods, such as EBER staining.

The term “short tandem repeat (STR) locus” refers to regions of a genome which contains short, repetitive sequence elements of 2 to 7 base pairs in length. Each sequence element is repeated at least once within an STR and is referred to herein as a “repeat unit.” The term STR also encompasses a region of genomic DNA wherein more than a single repeat unit is repeated in tandem or with intervening bases, provided that at least one of the sequences is repeated at least two times in tandem. Examples of STRs, include but are not limited to, a triplet repeat, e.g., ATC in tandem; a 4-peat (tetra-repeat), e.g., GATA in tandem; and a 5-peat (penta-repeat), e.g., ATTGC in tandem and so on. Information about specific STRs that can be used as genetic markers can be found in, among other places, the STRbase at www.cstl.nist.gov/strbase or https://web.expasy.org/cellosaurus-str-search/.

In one exemplary embodiment, the human nasopharyngeal carcinoma cells described herein are metastatic human nasopharyngeal carcinoma cells. Non limiting examples of human metastatic human nasopharyngeal carcinoma cells are metastatic cells of the bone, liver, lung or soft tissue.

In one exemplary embodiment, the human nasopharyngeal carcinoma cells described herein express at least one of the following: EGFR (epidermal growth factor receptor), RB1, E2F1, CC ND1, CDK4, CDK6, PCNA, ERK, or AKT.

The present invention also provides PDX derived human nasopharyngeal carcinoma cells designated as NPC-B13 (deposited at The American Type Culture Collection (ATCC), 10801 University Boulevard, Manassas, VA 20110, USA, under ATCC Accession Number PTA-127019).

Also provided are cellular compositions comprising a plurality of human nasopharyngeal carcinoma cells described herein.

The present invention further discloses in vitro methods for identifying a test therapeutic agent to inhibit NPC cells, comprising the steps of : a) contacting the test therapeutic agent with a specific quantity of NPC cells described herein; and b) determining the quantify of NPC cells or half maximal inhibitory concentration level (IC50) of the test therapeutic agent, wherein after contacting the test therapeutic agent with the NPC cells described herein, the quantify of NPC cells remains the same or reduced compared to the quantify of NPC prior to the contact, or the therapeutic effective IC50 is reached, is an indication that the test therapeutic agent is efficacious for treating NPC.

Embodiments of the present invention are illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the invention. During the studies described in the following examples, conventional procedures were followed, unless otherwise stated. Some of the procedures are described below for illustrative purpose.

### Description of Materials and Methods Used in the Examples

Materials and methods

Drugs: Palbociclib (Ibrance) was purchased from Pfizer Manufacturing Deutschland GmbH (Freiburg, Germany). Afatinib (Giotrief) was purchased from Boehringer Ingelheim Pharma GmbH & Co. KG (Ingelheim am Rhein, Germany) and cetuximab (Erbitux) was procured from Merck Healthcare KGaA (Darmstadt, Germany).

Cell Growth Assay and Animal Studies

NPC cell lines, C666-1 (EBV positive) and HK-1 (EBV negative), were maintained in RPMI containing 10% fetal bovine serum (FBS). A cell growth assay and animal studies were conducted as described by CL Hsu et al (“Application of a Patient-Derived Xenograft Model in Cytolytic Viral Activation Therapy for Nasopharyngeal Carcinoma.” Oncotarget 6, no. 31 (2015): 31323-34). All experiments involving laboratory animals followed the guidelines for animal experiments of Chang Gung Memorial Hospital (CGMH) and were approved by the IACUC of CGMH.

### EXAMPLE 1

### NPC PDX Establishment

PDX models were generated as follows: NPC tumor samples were obtained from patients with local recurrence or metastasis undergoing biopsy or surgical resection. Each sample was immediately cut into smaller pieces of approximately 3-5 mm in diameter and subcutaneously implanted into the flank regions of anesthetized NOD/SCID mice. The xenograft was excised and reimplanted in small pieces into the next passage after the tumor reached 1-2 cm in diameter. The nomenclature system for our NPC PDX was NPC PDX-metastasized tissue (abbreviation)-patient number. Accordingly, the NPC PDX-ST1 and PDX-B13 corresponded to NPC PDX-soft tissue and NPC PDX-bone in reference, see Table 1 below. Please also see HP Li et al, entitle “Combination of Epithelial Growth Factor Receptor Blockers and CDK4/6 Inhibitor for Nasopharyngeal Carcinoma Treatment” Cancers 2021, 13, 2954, the entire disclosure is incorporated herein by reference.

TABLE 1

| NPC PDX | | |
|---|---|---|
| Designation | Metastatic site | Patient ID No. |
| NPC PDX-ST1 | Soft Tissue | 1 |
| NPC PDX-Lu3 | Lung | 3 |
| NPC PDX-Li11 | Liver | 11 |
| NPC PDX-B13 | Bone | 13 |
| NPC PDX-B33 | Bone | 33 |
| NPC PDX-Li41 | Liver | 41 |

### EXAMPLE 2

### The Establishment of PDX derived NPC-B13 cell line

NPC-B13 PDX of Example 1 was cut into pieces, and cells were then dissociated through repetitive pipetting in culture medium. Dissociated cells were pelleted through centrifugation and cultured in Dulbecco's modified Eagle medium (Nutrient Mixture F-12 [DMEM/F12]; Gibco) supplemented with 1% FBS, 2μM hydrocortisone (Sigma), 1% N2 supplement (Gibco), 1% insulin—transferrin—selenium (Gibco), 20 ng/mL human EGF (Gibco), 2 mM L-glutamine (Gibco), and 100 U/mL penicillin—streptomycin (Gibco). Although the NPC-B13 cell line was passaged for more than 80 passages in vitro, it did not form a xenograft in NOD/SCID mice.

### Example 3

### The Short Tandem Repeats (STR) of PDX derived NPC-B13 cell line of Example 2

The DNA of NPC-B13 cell line of Example 2 was extracted (Conc.: 522 ng/μl; OD260/280: 1.97) and purified (conc=115.8 ng/μl; OD260/280=2.03; OD260/230=2.11. The STR loci were amplified by Promega GenePrint® 24 System. The CE analysis was performed on ABI PRISM 3730 GENETIC ANALYZER and the raw data was analyzed by GeneMapper® Software V3.7. The Short Tandem Repeat loci and DNA typing of the PDX derived NPC-B13 cell line of Example 2 are listed in Table 2 and Table 3 below.

TABLE 2
STR Analysis Result

| ANSI/ATCC | | | | | |
|---|---|---|---|---|---|
| ASN-0002 | Repeat | Extended | Repeat | Extended | Repeat |
| STR Loci | Numbers | STR Loci | Numbers | STR Loci | Numbers |
| D5S818 | 11, 11 | D3S1358 | 16, 16 | DYS391 | N/A |
| D13S317 | 8, 8 | D1S1656 | 11, 16, 17 | D8S1179 | 12, 12 |
| D7S820 | 11, 11 | D2S441 | 12, 14 | D12S391 | 19, 23 |
| D16S539 | 9, 9 | D10S1248 | 13, 15 | D19S433 | 14, 14.2 |
| vWA | 14, 16 | PentaE | 15, 15 | FGA | 27, 27 |
| TH01 | 7, 7 | D18S51 | 15, 15 | D22S1045 | 16, 16 |
| Amelogenin | X | D2S1338 | 18, 23 | | |
| TPOX | 8, 11 | PentaD | 8, 12 | | |
| CSF1PO | 10, 11 | D21S11 | 29, 30 | | |

TABLE 3
DNA Typing Analysis Result

| STR Loci | Allele | Height | Area |
|---|---|---|---|
| AMEL | X | 81.98 | 7161 |
| D3S1358 | 16 | 128.37 | 7651 |
| D1S1656 | 11 | 167.97 | 19817 |
| D1S1656 | 16 | 188.25 | 11443 |
| D1S1656 | 17 | 192.28 | 10450 |
| D2S441 | 12 | 228.03 | 5886 |
| D2S441 | 14 | 236.27 | 5723 |
| D10S1248 | 13 | 272.07 | 5971 |
| D10S1248 | 15 | 279.92 | 6080 |
| D13S317 | 8 | 312.07 | 6436 |
| Penta E | 15 | 416.9 | 8821 |
| D16S539 | 9 | 97.41 | 11897 |
| D18S51 | 15 | 166.45 | 11056 |
| D2S1338 | 18 | 255.7 | 5176 |
| D2S1338 | 23 | 276.21 | 5271 |
| CSF1PO | 10 | 336.97 | 8525 |
| CSF1PO | 11 | 341.01 | 4856 |
| Penta D | 8 | 410.1 | 3442 |
| Penta D | 12 | 430.81 | 3135 |
| TH01 | 7 | 85.35 | 2522 |
| vWA | 14 | 144.59 | 3313 |
| vWA | 16 | 152.71 | 4995 |
| D21S11 | 29 | 225.54 | 3995 |
| D21S11 | 30 | 229.67 | 4703 |
| D7S820 | 11 | 296.63 | 10045 |
| D5S818 | 11 | 344.74 | 5377 |
| TPOX | 8 | 411.99 | 1799 |
| TPOX | 11 | 424.09 | 1577 |
| D8S1179 | 12 | 96.42 | 8255 |
| D12S391 | 19 | 158.45 | 5546 |
| D12S391 | 23 | 175.08 | 3916 |
| D19S433 | 14 | 228.35 | 2829 |
| D19S433 | 14.2 | 230.37 | 2535 |
| FGA | 27 | 317.36 | 2644 |
| D22S1045 | 16 | 459.28 | 3129 |

As illustrated in Table 2 and Table 3, the PDX derived NPC-B13 cell line of Example 2 comprises the following short tandem repeat loci: D5S818, D13S317, D7S820, D16S539, vWA, TH01, Amelogenin, TPDX, CSF1PO, D3S1358, D1S1656, D2S441, D10S1248, Penta E, D18S51, D2S1338, Penta D, D21S11, DYS19, D8S1179, D12S391, D19S433, FGA and D22S1045.

The sequences of the STR loci can be found at the STR database of National institute of standards and technology (https://strbase.nisi.gov/), the sequence of each STR locus identified in the STR database of National institute of standards and technology is incorporated herein by reference.

### EXAMPLE 4

### Cell Morphology and EBV Staining of PDX derived NPC-B13 cell line of Example 2

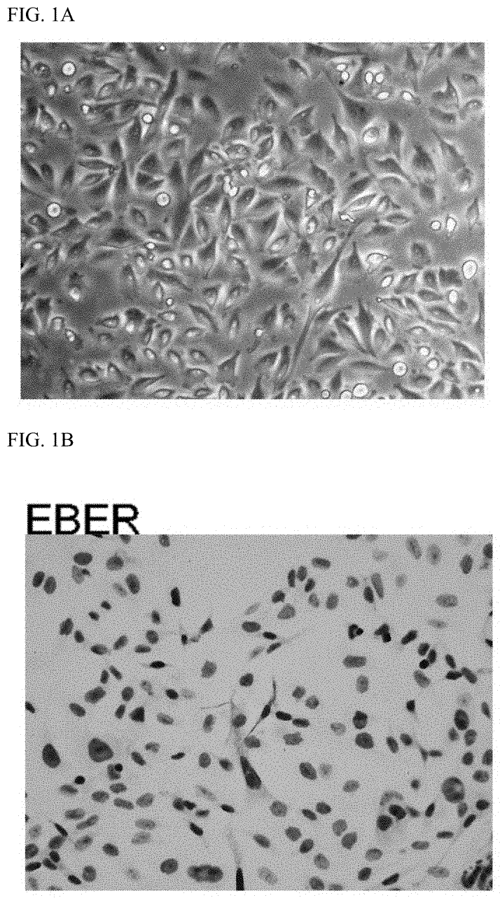

FIG. 1A is a phase microscopic image (400X) of the of PDX derived NPC-B13 cell line of Example 2. FIG. 1B is the EBER staining of EBV-encoded RNA and illustrates the presence of EBV in the PDX derived NPC-B13 cell line of Example 2.

### EXAMPLE 5

### Western Blot Analysis of EGFR

One hundred micrograms of the protein lysate per lane was used for Western blot analysis. Antibodies used in this study were EGFR (Santa Cruz SC-373746) and beta-actin.

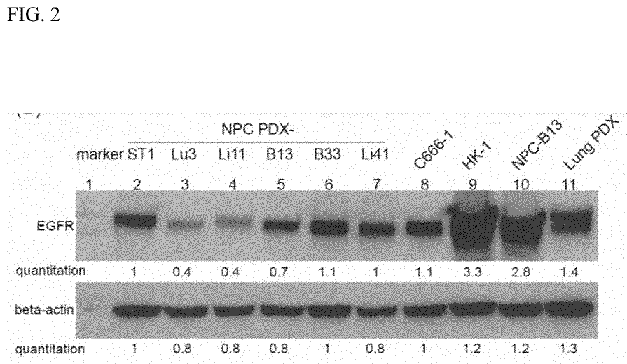

FIG. 2 shows the Western blot analysis of EGFR expression in six PDX derived NPC cells listed in Table 1 (lanes 2-7), three NPC cells (lane 8: C666-1, lane 9: HK-1 and lane 10: NPC-B13) and PDX derived lung cancer cells (lane 11, EGFR-positive control). EGFR protein was highly expressed in HK1 and NPC-B13 cells.

### EXAMPLE 6

### The use of the PDX derived NPC-B13 Cell Line of Example 2 to Identify Potential Therapeutic Agent

Food and Drug Administration—approved EGFR-targeted therapeutics certuximab (Erbitux, ERB) and palbociclib (PAL), which have been reported to suppress NPC, were used to determine if the PDX derived NPC cells of Example 2 can be used to identify potential therapeutic agent for NPC.

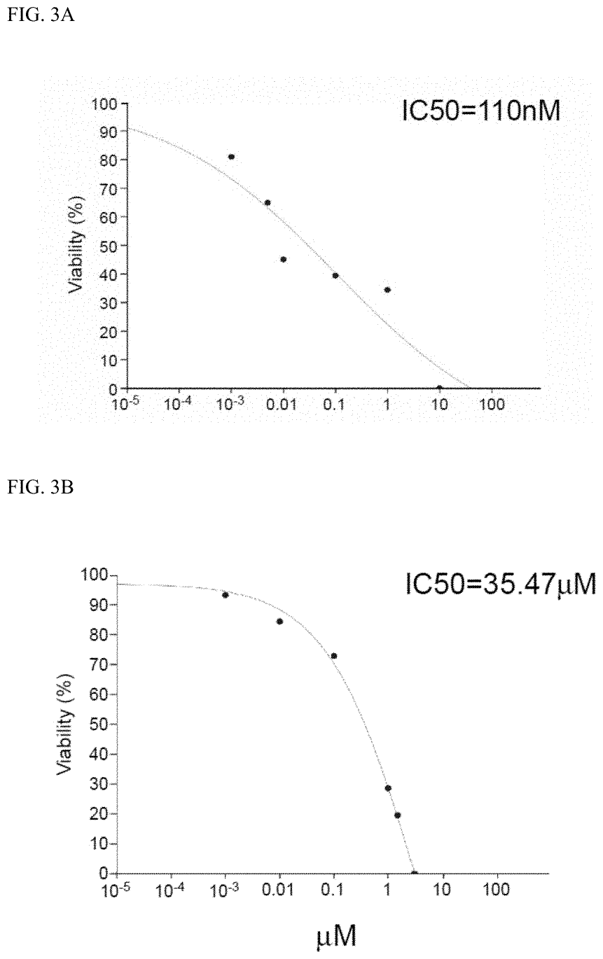

Briefly, the PDX derived NPC cells of Example 2 were cultured with ERB and PAL. FIG. 3A and FIG. 3B show the IC₅₀ (IC50 Calculator, AAT Bioquest) of ERB and PAL in the PDX derived NPC cells of Example 2.

### EXAMPLE 7

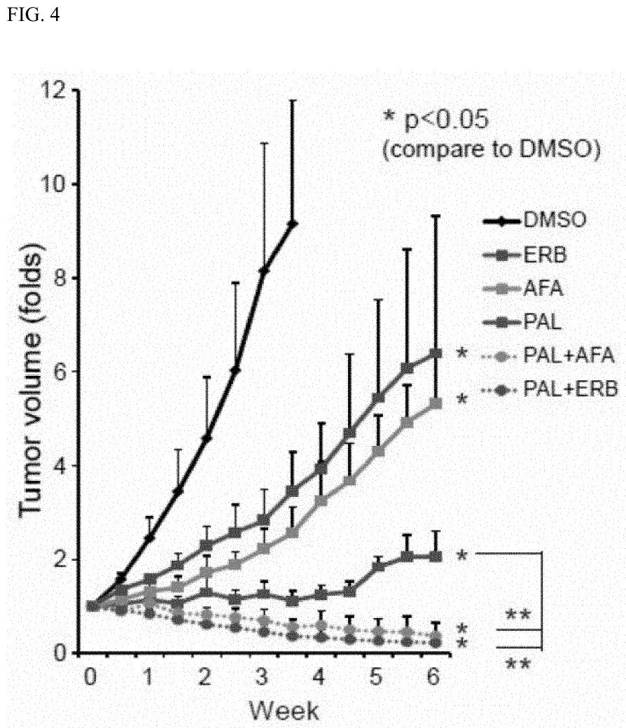

An in vivo study was performed to examine the effect of EGFR inhibitors, cetuximab (ERB) and afatinib (AFA), and a cell cycle blocker, palbociclib (PAL) on the tumor volume of NPC PDX-B13 mouse model of Example 1. As shown in FIG. 4, when EGFRi and PAL were applied separately, each of them could inhibit PDX-B13 tumor growth (reduce tumor volume) by approximately 30% and 70% on week 6. The combination of PAL+AFA and the combination of PAL+ERB could inhibit PDX-B13 tumor growth (reduce tumor volume) by approximately 90% on week 6.

What is claimed is:

1. A plurality of human nasopharyngeal carcinoma cells derived from a patient derived xenograft, said cells comprise:
(a) human herpesvirus 4 (HHV-4); and
(b) at least one of the following short tandem repeat locus: D5S818, D13S317, D7S820, D16S539, vWA, TH01, Amelogenin, PDX, CSF1PO, D3S1358, D1S1656, D2S441, D10S1248, PentaE, D18S51, D2S1338, PentaD, D21S11, DYS19, D8S1179, D12S391, D19S433, FGA or D22S1045.

2. The human nasopharyngeal carcinoma cells of claim 1, wherein the human nasopharyngeal carcinoma cells are metastatic nasopharyngeal carcinoma cells.

3. The human nasopharyngeal carcinoma cells of claim 1, wherein the human nasopharyngeal carcinoma cells express epidermal growth factor receptors.

4. The human nasopharyngeal carcinoma cells of claim 1, wherein the human nasopharyngeal carcinoma cells express at least one of the following: RB1, E2F1, CC ND1, CDK4, CDK6, PCNA, ERK, or AKT.

5. A plurality of human nasopharyngeal carcinoma cells derived from a patient derived xenograft, designated as NPC-B13 deposited under ATCC Accession Number PTA-127019.

6. A cellular composition, comprising the human nasopharyngeal carcinoma cells of claim 1

7. An in vitro methods for identifying a test therapeutic agent to inhibit NPC cells, comprising the steps of : a) contacting the test therapeutic agent with a plurality of the nasopharyngeal carcinoma cells of claim; and b) determining the quantify of the nasopharyngeal carcinoma cells or the half maximal inhibitory concentration level (IC50) of the test therapeutic agent, wherein after contacting the test therapeutic agent with the nasopharyngeal carcinoma cells, the quantify of the nasopharyngeal carcinoma cells remains the same or reduced compared to the quantify of NPC prior to the contact, or the therapeutic effective IC50 is reached, is an indication that the test therapeutic agent is efficacious for treating nasopharyngeal carcinoma.
